(19) United States
(12) Patent Application Publication
(10) Pub. No.: US 2023/0079269 A1
(43) Pub. Date: Mar. 16, 2023

(54) METHOD AND SYSTEM FOR EVALUATING STATUS OF FISTULA

(71) Applicant: Finedar Biomedical Technology Co. Ltd., Hsinchu City (TW)

(72) Inventors: Teh-Ho TAO, Zhunan Township (TW); Kent-Hao HWANG, Taoyuan City (TW)

(73) Assignee: Finedar Biomedical Technology Co. Ltd., Hsinchu City (TW)

(21) Appl. No.: 17/890,015

(22) Filed: Aug. 17, 2022

(30) Foreign Application Priority Data
Aug. 27, 2021 (TW) 110131859

(51) Int. Cl. A61B 5/0265; A61B 5/05; A61B 5/00

(52) U.S. Cl. CPC A61B 5/0265; A61B 5/05; A61B 5/7267; A61B 5/7257; A61B 5/002

(57) ABSTRACT

A system for evaluating a status of a fistula of a subject includes a radio device that emits a carrier radio wave toward the fistula, and that receives a return wave signal formed through reflection of the carrier radio wave by the fistula, and an evaluating device that performs a time-frequency transform on a digitized detection signal related to the return wave signal to result in frequency spectrum information, that calculates a magnitude ratio based on the frequency spectrum information, that generates an evaluation result by using a machine learning model based on the magnitude ratio, and that outputs the evaluation result which indicates the status of the fistula.

### CROSS-REFERENCE TO RELATED APPLICATION

This application claims priority to Taiwanese Invention Patent Application No. 110131859, filed on Aug. 27, 2021.

### FIELD

The disclosure relates to a method and a system for medical evaluation, and more particularly to a method and a system for evaluating a status of a fistula.

### BACKGROUND

An arteriovenous (AV) fistula is usually surgically created in a patient with chronic kidney disease for allowing the patient to receive kidney dialysis. Since a blood flow rate in the AV fistula is highly related to efficiency of kidney dialysis, once AV fistula occlusion occurs in a patient, the kidney dialysis may be ineffective or not performable, which may result in the need for emergency treatment or even hospitalization of the patient.

Conventionally, HD03 hemodialysis monitors produced by Transonic Systems Inc. are used for blood flow evaluation in AV fistula. However, such conventional evaluation approach is intrusive and requires inserting two needles into the blood vessel(s) of a patient. Moreover, while able to provide a high degree of accuracy, such conventional evaluation approach is costly because of high-priced equipment (i.e., the HD03 hemodialysis monitors) and consumables (e.g., needles or tubing). For this reason, it is unsuitable for regular check-ups.

### SUMMARY

Therefore, an object of the disclosure is to provide a system and a method that are adapted for evaluating a status of a fistula of a subject and that can alleviate at least one drawback of the prior art.

According to one aspect of the disclosure, the system includes a radio device and an evaluating device.

The radio device includes a transmitting antenna, a receiving antenna, a transmitting module that is configured to cooperate with said transmitting antenna to emit a carrier radio wave toward the fistula, and a receiving module that is configured to receive, via said receiving antenna, a return wave signal that is formed through reflection of the carrier radio wave by the fistula, and to output a transmission signal that is generated based on the return wave signal.

The evaluating device includes a communication module, a time-frequency transform module, a calculating module and an evaluating module. The communication module is in signal connection with said receiving module, and is configured to receive the transmission signal, and to recover a digitized detection signal from the transmission signal. The time-frequency trans form module receives the digitized detection signal from said communication module, and performs a time-frequency transform on the digitized detection signal to result in a frequency spectrum information, and to output the frequency spectrum information. The calculating module is configured to receive the frequency spectrum information from the time-frequency transform module, to determine, from the frequency spectrum information, a frequency that corresponds to a greatest magnitude as a fundamental frequency, to calculate at least a magnitude of harmonic, and to calculate at least one magnitude ratio that is one of following items:

a ratio of one of the at least one magnitude of harmonic to the greatest magnitude that corresponds to the fundamental frequency;

a ratio of one of the at least one magnitude of harmonic to another one of the at least one magnitude of harmonic; and

a combination thereof, where the at least one magnitude of harmonic is a peak magnitude for at least one harmonic frequency band with respect to the fundamental frequency.

The evaluating module is in signal connection with said calculating module, and is configured to receive the at least one magnitude ratio thus calculated, to generate an evaluation result by using a machine learning model based on the at least one magnitude ratio, and to output the evaluation result that indicates the status of the fistula.

According to another aspect of the disclosure, the method includes following steps. In one step, a carrier radio wave is emitted toward the fistula. In one step, a return wave signal that is formed through reflection of the carrier radio wave by the fistula is received, and a transmission signal that is generated based on the return wave signal is outputted. In one step a digitized detection signal is recovered from the transmission signal. In one step, a time-frequency transform is performed on the digitized detection signal to result in frequency spectrum information, and the frequency spectrum information is outputted. In one step, a frequency that corresponds to a greatest magnitude is determined from the frequency spectrum information as a fundamental frequency, at least one magnitude of harmonic is calculated, and at least one magnitude ratio is calculated. The at least one magnitude ratio is one of following item:
a ratio of one of the at least one magnitude of harmonic to the greatest magnitude that corresponds to the fundamental frequency;
a ratio of one of the at least one magnitude of harmonic to another one of the at least one magnitude of harmonic; and
a combination thereof, where the at least one magnitude of harmonic is a peak magnitude for at least one harmonic frequency band with respect to the fundamental frequency.

In one step, an evaluation result is generated by using a machine learning model based on the at least one magnitude ratio, and the evaluation result that indicates the status of the fistula is outputted.

### BRIEF DESCRIPTION OF THE DRAWINGS

Other features and advantages of the disclosure will become apparent in the following detailed description of the embodiment(s) with reference to the accompanying drawings, of which:

FIG. 1 is a schematic diagram illustrating a radio device to be disposed on a subject according to an embodiment of the disclosure;

FIG. 2 is a block diagram illustrating a system for evaluating a status of a fistula of a subject according to an embodiment of the disclosure;

FIG. 3 illustrates a waveform of a digitized detection signal for a healthy subject according to an embodiment of the disclosure;

FIG. 4 illustrates a waveform of a digitized detection signal for a subject with a stenotic fistula according to an embodiment of the disclosure;

FIG. 5 illustrates a frequency spectrum information obtained from the digitized detection signal of FIG. 3 after filtering process and time-frequency transform;

FIG. 6 illustrates a frequency spectrum information obtained from the digitized detection signal of FIG. 4 after filtering process and time-frequency transform;

FIG. 7 is a flow chart illustrating a method for evaluating a status of a fistula of a subject according to an embodiment of the disclosure;

FIG. 8 is a flow chart illustrating steps related to a training process according to an embodiment of the disclosure; and

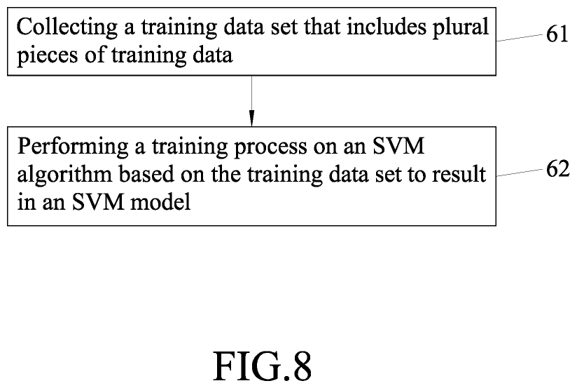

FIG. 9 illustrates an example of a curve of receiver operating characteristic (ROC) according to an embodiment of the disclosure.

### DETAILED DESCRIPTION

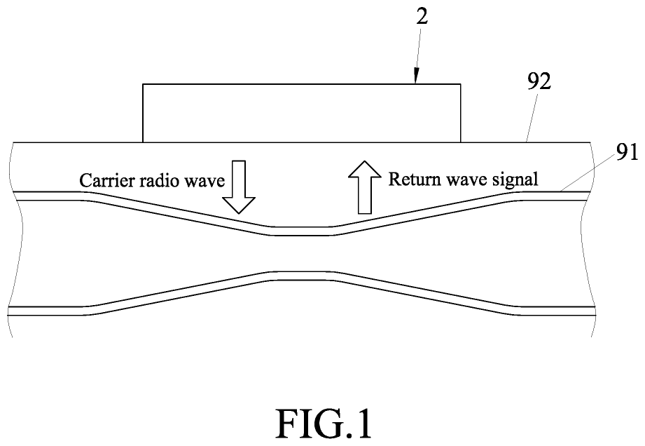

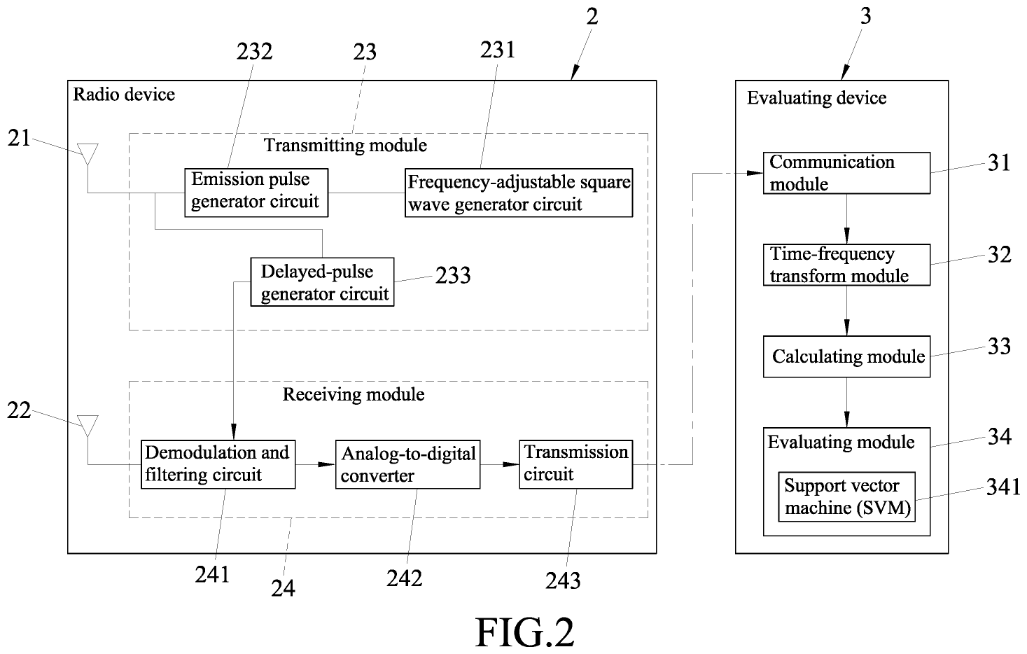

Referring to FIGS. 1 and 2, a system according to an embodiment of this disclosure is adapted for evaluating a status of a fistula, such as an arteriovenous (AV) fistula, of a subject. The system includes a radio device 2 and an evaluating device 3.

The radio device 2 is configured to be disposed on the skin 92 of the subject above a fistula 91. For example, the radio device 2 may be disposed on the skin 92 at a position adjacent to a venous side of the fistula 91. The radio device 2 includes a transmitting antenna 21, a receiving antenna 22, a transmitting module 23 and a receiving module 24. The transmitting module 23 is configured to cooperate with the transmitting antenna 21 to emit a carrier radio wave toward the fistula 91. The receiving module 24 is configured to receive, via the receiving antenna 22, a return wave signal that is formed through reflection of the carrier radio wave by the fistula 91, and to output a transmission signal that is generated based on the return wave signal.

Specifically, the transmitting module 23 includes a frequency-adjustable square wave generator circuit 231, an emission pulse generator circuit 232 and a delayed-pulse generator circuit 233. The emission pulse generator circuit 232 is electrically connected to and disposed between the transmitting antenna 21 and the frequency-adjustable square wave generator circuit 231. The delayed-pulse generator circuit 233 is electrically connected to the transmitting antenna 21. The receiving module 24 includes a demodulation and filtering circuit 241, an analog-to-digital converter 242 and a transmission circuit 243. The demodulation and filtering circuit 241 is electrically connected to the receiving antenna 22 and the delayed-pulse generator circuit 233. The analog-to-digital converter 242 is electrically connected to the demodulation and filtering circuit 241. The transmission circuit 243 is electrically connected to the analog-to-digital converter 242 and outputs the transmission signal. In some embodiments, transmission of the transmission signal by the transmission circuit 243 is implemented based on the Bluetooth wireless technology.

Details regarding hardware configurations and operating principles of the radio device 2 can be found in U.S. Patent Application Publication No. 20210038093, the disclosure of which is incorporated herein by reference. Therefore, the details are not repeated herein for the sake of brevity.

The evaluating device 3 includes a communication module 31, a time-frequency transform module 32, a calculating module 33 and an evaluating module 34. In some embodiments, the communication module 31, the time-frequency transform module 32 and the calculating module 33 are implemented together by a portable electronic device (e.g., a smartphone or a tablet), and more specifically, a transceiver, a microprocessor and/or a digital signal processor included in the portable electronic device; and the evaluating module 34 is implemented by a server. In some embodiments, a smartphone is installed with a mobile application (app) to carry out functions of the communication module 31, the time-frequency transform module 32 and the calculating module 33. However, implementation of the evaluating device 3 is not limited to the disclosure herein and may vary in other embodiments. For example, the evaluating device 3 may be implemented entirely by a single server.

The communication module 31 of the evaluating device 3 is in signal connection with the receiving module 24 of the radio device 2. The communication module 31 is configured to receive the transmission signal, and to recover a digitized detection signal from the transmission signal. In some embodiments, receipt of the transmission signal by the communication module 31 is implemented based on the Bluetooth wireless technology.

The time-frequency transform module 32 is connected to the communication module 31, and is configured to receive the digitized detection signal, to perform a time-frequency transform on the digitized detection signal to result in frequency spectrum information, and to output the frequency spectrum information. Specifically, the time-frequency transform module 32 is configured to perform a filtering process on the digitized detection signal to result in a filtered signal that is in a specific passband, and to perform the time-frequency transform on the filtered signal to result in the frequency spectrum information. In some embodiments, the time-frequency transform module 32 may be a software module. In some embodiments, the filtering process is a digital filtering process, and is implemented by using a finite impulse response (FIR) filter. Specifically, the FIR filter allows components of the digitized detection signal in the specific passband to pass through and blocks the remainder of the digitized detection signal outside the specific passband (i.e., bandpass filtering) to result in the filtered signal. In some embodiments, the specific passband ranges from about 0.2 Hz to about 10 Hz. In some embodiments, the time-frequency transform is implemented by a fast Fourier transform (FFT), and the time-frequency transform module 32 performs the FFT on the filtered signal to obtain the frequency spectrum information.

In some embodiments, the time-frequency transform module 32 stores in the evaluating device 3 (e.g., a smartphone) the digitized detection signal that is received from the communication module 31 and that would contain information on periodic movement and displacements of the fistula 91, and the digitized detection signal is displayed on a screen of the evaluating device 3 for viewing by a user.

The calculating module 33 is connected to the time-frequency transform module 32, and is configured to receive the frequency spectrum information, to determine, from the frequency spectrum information, a frequency that corresponds to a greatest magnitude as a fundamental frequency, to calculate at least one magnitude of harmonic, and to calculate at least one magnitude ratio. The at least one magnitude of harmonic is a peak magnitude for at least one harmonic frequency band with respect to the fundamental frequency. Specifically, each of the at least one harmonic frequency band is a specific range of frequencies where a center frequency of the specific range of frequencies is a corresponding harmonic that is an integer multiple of the fundamental frequency. The peak magnitude for each of the at least one harmonic frequency band is the greatest magnitude among magnitudes that correspond to frequencies in the specific range (i.e., a local maximum within the harmonic frequency band). The at least one magnitude ratio is one of the following items: (i) a ratio of one of the at least one magnitude of harmonic to the greatest magnitude that corresponds to the fundamental frequency, (ii) a ratio of one of the at least one magnitude of harmonic to another one of the at least one magnitude of harmonic, and (iii) a combination of items (i) and (ii).

For example, the calculating module 33 is configured to calculate the at least one magnitude of harmonic that includes n−1 number of magnitudes of harmonics, the at least one harmonic frequency band includes n−1 number of harmonic frequency bands with respect to the fundamental frequency, and the at least one magnitude ratio includes n−1 number of magnitude ratios. The n−1 number of magnitudes of harmonics are peak magnitudes respectively for the n−1 number of harmonic frequency bands. Specifically, the calculating module 33 is configured to select the n−1 number of harmonic frequency bands from within the specific passband (e.g., 0.2 Hz - 10 Hz) before calculating the peak magnitudes respectively for the n−1 number of harmonic frequency bands. The calculating module 33 then calculates the n−1 number of magnitude ratios based on an expression:

$$\frac{{P}_{i}}{{P}_{i - 1}} ,$$

where i represents an integer variable that starts from two to n, n represents a positive integer greater than one, Pᵢ represents an i^{th} magnitude of harmonic of the n−1 number of magnitudes of harmonics, Pᵢ₋₁ represents an (i−1)^{th} magnitude of harmonic of the n−1 number of magnitudes of harmonics, and P₁ represents the greatest magnitude that corresponds to the fundamental frequency (i.e., a magnitude that corresponds to the 1^{st} harmonic, and may be referred to as “fundamental frequency magnitude” hereinafter).

In another example, the calculating module 33 may calculate only one magnitude ratio, such as

$$\frac{{P}_{2}}{{P}_{1}}$$

(i.e., the ratio of the second magnitude of harmonic to the fundamental frequency magnitude),

$$\frac{{P}_{3}}{{P}_{1}}$$

(i.e., the ratio of the third magnitude of harmonic to the fundamental frequency magnitude),

$$\frac{{P}_{4}}{{P}_{1}}$$

(i.e., the ratio of the fourth magnitude of harmonic to the fundamental frequency magnitude),

$$\frac{{P}_{5}}{{P}_{4}}$$

(i.e., the ratio of the fifth magnitude of harmonic to the fourth magnitude of harmonic),

$$\frac{{P}_{3}}{{P}_{2}}$$

(i.e., the ratio of the third magnitude of harmonic to the second magnitude of harmonic), or

$$\frac{{P}_{4}}{{P}_{2}}$$

(i.e., the ratio of the fourth magnitude of harmonic to the second magnitude of harmonic). Alternatively, the calculating module 33 may calculate the n−1 number of magnitude ratios based on an expression of

$$\frac{{P}_{i - 1}}{{P}_{i}} ,$$

with the same definitions as

$$\frac{{P}_{i}}{{P}_{i - 1}} .$$

The evaluating module 34 is in signal connection with the calculating module 33, and is configured to receive the at least one magnitude ratio thus calculated, to generate an evaluation result by using a machine learning model based on the at least one magnitude ratio, and to output the evaluation result that indicates the status of the fistula. For example, the evaluating module 34 is configured to generate the evaluation result by using the machine learning model based on the n−1 number of magnitude ratios, and to output the evaluation result. In some embodiments, the evaluating module 34 is configured to store a support vector machine (SVM) model 341 to serve as the machine learning model, and to generate the evaluation result by using the SVM model 341 based on the n−1 number of magnitude ratios.

In some embodiments, the evaluating module 34 and the calculating module 33 are capable of communicating with each other by using wireless communication or networking technologies, such as global system for mobile communications (GSM), other generations of wireless mobile telecommunications technology, Wi-Fi, Bluetooth or the like.

In some embodiments, the evaluating module 34 stores a plurality of SVM models each of which corresponds to a respective one of different blood flow parameters, and which are obtained in advance by using a training process that includes a cross-validation technique (e.g., k-fold cross-validation).

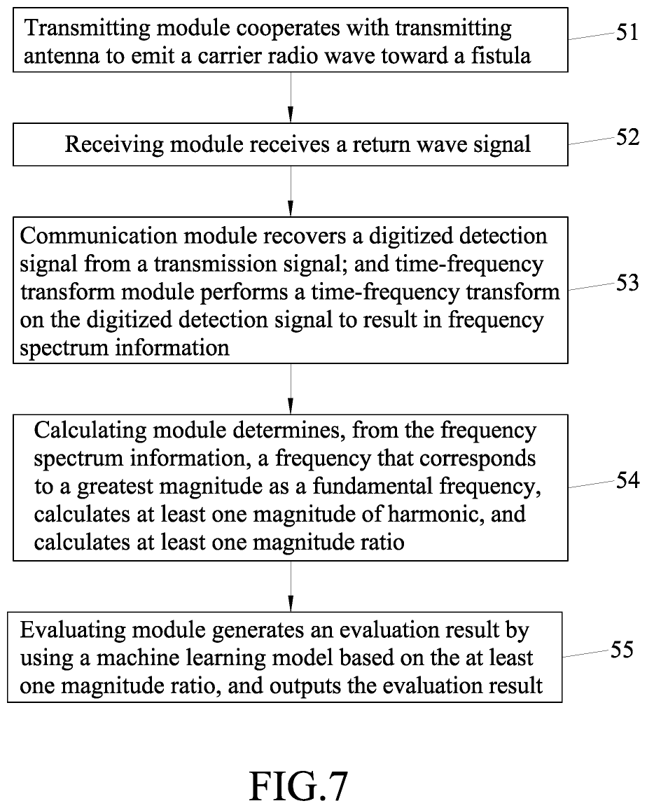

Referring to FIG. 7, in practice, a method according to an embodiment of this disclosure is adapted for evaluating a status of a fistula, and may be implemented by using the system exemplarily shown in FIGS. 1 and 2. The system includes following steps 51 to 55.

In step 51, the transmitting module 23 cooperates with the transmitting antenna 21 to emit a carrier radio wave toward the fistula 91.

In step 52, the receiving module 24 receives, via the receiving antenna 22, a return wave signal that is formed through reflection of the carrier radio wave by the fistula 91, and to output a transmission signal that is generated based on the return wave signal.

In step 53, the communication module 31 of the evaluating device 3 receives the transmission signal and recovers a digitized detection signal from the transmission signal, and the time-frequency transform module 32 of the evaluating device 3 performs a time-frequency transform on the digitized detection signal to result in frequency spectrum information and outputs the frequency spectrum information.

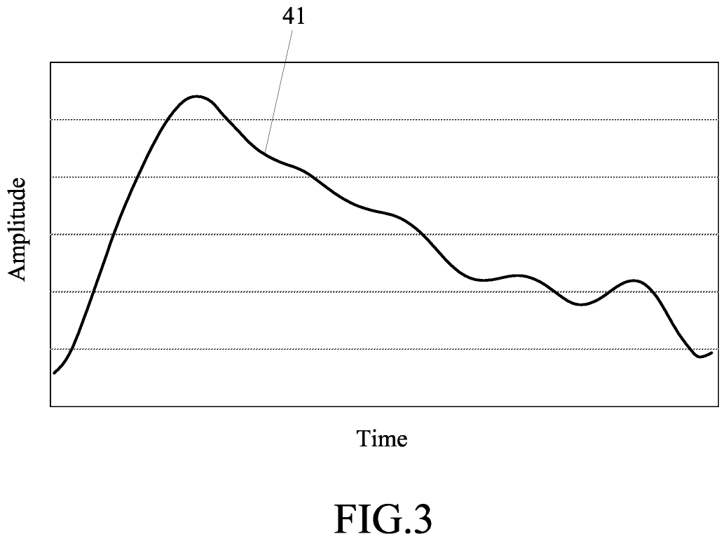

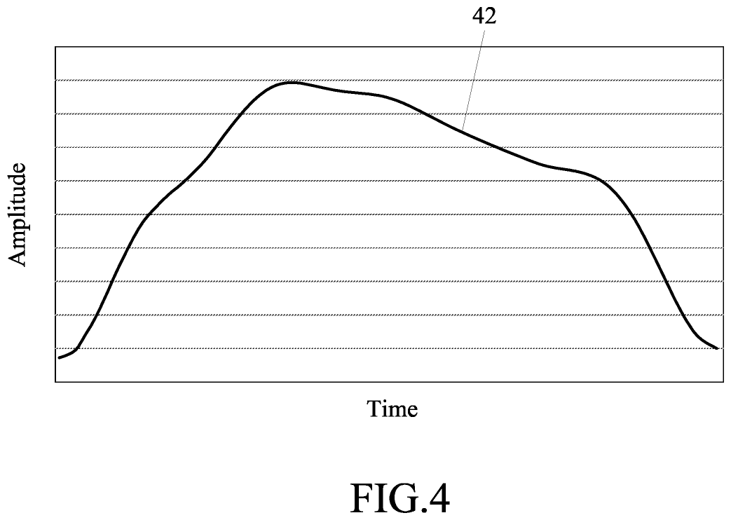

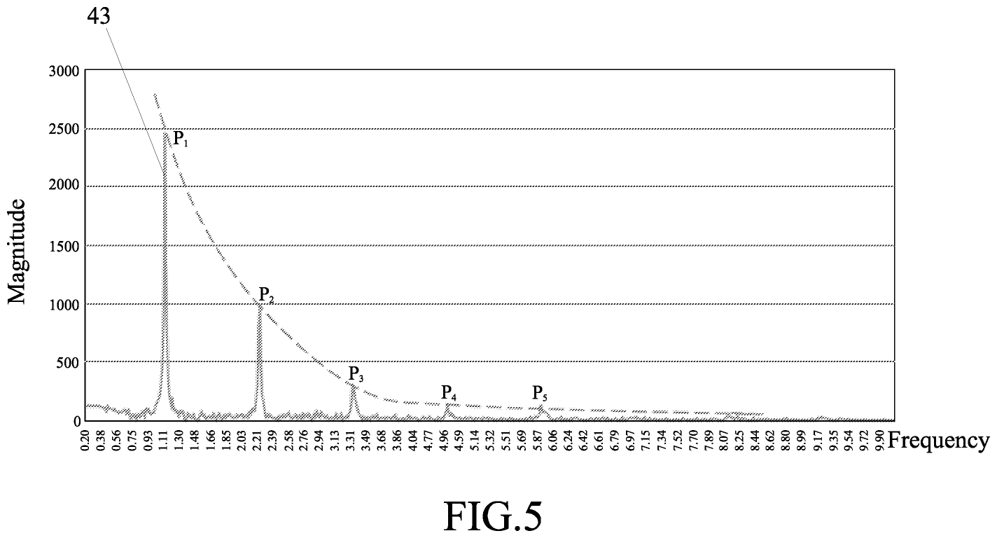

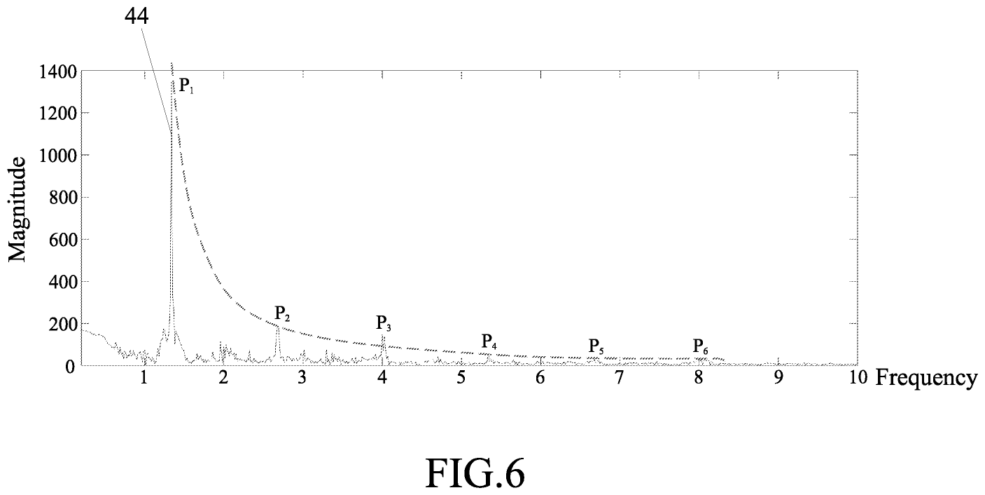

In some embodiments, the time-frequency transform module 32 first samples a one-minute segment of the digitized detection signal at a sampling rate of, such as but not limited to, 64 Hz. The time-frequency transform module 32 then performs a filtering process (e.g., a digital filtering process) on the digitized detection signal thus sampled to result in a filtered signal that is in a specific passband (e.g., about 0.2 Hz to about 10 Hz), and performs a time-frequency transform on the filtered signal to result in the frequency spectrum information. The digitized detection signal is a time domain signal. Referring to FIGS. 3 and 4, waveforms 41 and 42 of two examples of the digitized detection signal are illustrated. FIGS. 5 and 6 illustrate spectra 43 and 44 of two examples of the frequency spectrum information that are obtained from the two examples of FIGS. 3 and 4, respectively, after the filtering process and the time-frequency transform.

In step 54, the calculating module 33 receives the frequency spectrum information, determines, from the frequency spectrum information, a frequency that corresponds to a greatest magnitude as a fundamental frequency, calculates at least one magnitude of harmonic, and calculates at least one magnitude ratio. The at least one magnitude of harmonic is a peak magnitude for at least one harmonic frequency band with respect to the fundamental frequency. The at least one magnitude ratio is one of the following items: (i) a ratio of one of the at least one magnitude of harmonic to the greatest magnitude that corresponds to the fundamental frequency; (ii) a ratio of one of the at least one magnitude of harmonic to another one of the at least one magnitude of harmonic; and (iii) a combination of (i) and (ii).

In this embodiment, the calculating module 33 calculates the at least one magnitude of harmonic that includes n−1 number of magnitudes of harmonics, the at least one harmonic frequency band includes n−1 number of harmonic frequency bands with respect to the fundamental frequency, and the at least one magnitude ratio includes n−1 number of magnitude ratios. The n−1 number of magnitudes of harmonics are peak magnitudes respectively for the n−1 number of harmonic frequency bands. The calculating module 33 then calculates the n−1 number of magnitude ratios based on an expression:

$$\frac{{P}_{i}}{{P}_{i - 1}} ,$$

where i represents an integer variable that starts from two to n, n represents a positive integer greater than one, Pᵢ represents an i^{th} magnitude of harmonic of the n−1 number of magnitudes of harmonics, Pᵢ₋₁ represents an (i−1)^{th} magnitude of harmonic of the n−1 number of magnitudes of harmonics, and P₁ represents the greatest magnitude that corresponds to the fundamental frequency.

In other embodiments, the calculating module 33 may calculate only one magnitude ratio, such as

$$\frac{{P}_{2}}{{P}_{1}} , \frac{{P}_{3}}{{P}_{1}} , \frac{{P}_{4}}{{P}_{1}} , \frac{{P}_{5}}{{P}_{4}} , \frac{{P}_{3}}{{P}_{2}} , or \frac{{P}_{4}}{{P}_{2}} .$$

Alternatively, the calculating module 33 may calculate the at least one magnitude ratio that includes n−1 number of magnitude ratios based on the expression of

$$\frac{{P}_{i - 1}}{{P}_{i}} .$$

For example, taking n equaling two as an example, the calculating module 33 calculates a magnitude of harmonic that corresponds to a harmonic frequency band with respect to the fundamental frequency, such as a second harmonic frequency band with a center frequency being the 2^{nd} harmonic of the fundamental frequency. The calculating module 33 then calculates a magnitude ratio of

$$\frac{{P}_{2}}{{P}_{1}} ,$$

where P₂ is the magnitude of harmonic that corresponds to the second harmonic frequency band, and P₁ is the greatest magnitude that corresponds to the fundamental frequency.

On the other hand, taking n equaling six as another example, the calculating module 33 calculates five magnitudes of harmonics that respectively correspond to five harmonic frequency bands with respect to the fundamental frequency, such as a second harmonic frequency band, a third harmonic frequency band, a fourth harmonic frequency band, a fifth harmonic frequency band, and a sixth harmonic frequency band, each with a center frequency being a respective one of the 2^{nd} harmonic, the 3^{rd} harmonic, the 4^{th} harmonic, the 5^{th} harmonic and the 6^{th} harmonic of the fundamental frequency. The calculating module 33 then calculates magnitude ratios of

$$\frac{{P}_{2}}{{P}_{1}} , \frac{{P}_{3}}{{P}_{2}} , \frac{{P}_{4}}{{P}_{3}} , \frac{{P}_{5}}{{P}_{4}} and \frac{{P}_{6}}{{P}_{5}} ,$$

where P₂ to P₅ are the magnitudes of harmonics that respectively correspond to the second harmonic frequency band, the third harmonic frequency band, the fourth harmonic frequency band, the fifth harmonic frequency band and the sixth harmonic frequency band, and P₁ is the greatest magnitude that corresponds to the fundamental frequency.

Referring to FIG. 6, in some embodiments where the specific passband ranges from about 0.2 Hz to about 10 Hz, since a frequency to which the greatest magnitude corresponds (i.e., the fundamental frequency) is usually within a range of about 1 Hz to about 2 Hz, n is generally set as a positive integer smaller than ten, or even smaller than eight. In this embodiment, the calculating module 33 calculates the at least one magnitude of harmonic and the at least one magnitude ratio with n being set to six.

In some embodiments, the magnitudes of harmonics P₂ to Pₙ are calculated in the following manner. First, the fundamental frequency is multiplied by two to n to obtain n−1 number of harmonics (i.e., the 2^{nd} harmonic to the n^{th} harmonic). Then, for each of the n−1 number of harmonics, a frequency band that is a specific range of frequencies with a center frequency being the harmonic is determined as a respective one of the n−1 number of harmonic frequency bands. Next, for each of the n−1 number of harmonic frequency bands, the greatest magnitude among magnitudes that correspond to frequencies in the specific range (i.e., a local maximum within the harmonic frequency band) is determined as the peak magnitude for the harmonic frequency band (i.e., a respective one of the n−1 number of magnitudes of harmonics). Taking the second magnitude of harmonic as an example, if the fundamental frequency is 1.3 Hz and a width of each harmonic frequency band is 0.1 Hz, the fundamental frequency is multiplied by two (i.e., the 2^{nd} harmonic) to obtain 2.6 Hz as a center frequency, and the specific range of frequencies 2.55 Hz to 2.65 Hz is determined as the second harmonic frequency band. After that, the greatest magnitude among magnitudes that correspond to frequencies in the specific range 2.55 Hz to 2.65 Hz is determined as the second magnitude of harmonic.

In step 55, the evaluating module 34 receives the at least one magnitude ratio thus calculated, generates an evaluation result by using a machine learning model based on the at least one magnitude ratio, and outputs the evaluation result that indicates the status of the fistula 91 as to whether a blood flow rate in the fistula 91 is greater than a particular flow rate parameter that corresponds to the machine learning model.

In some embodiments, the evaluating module 34 generates the evaluation result by using the SVM model 341 based on the n−1 number of magnitude ratios, and outputs the evaluation result. The evaluation result is a category predicted by the SVM model 341, and is one of 1 and 0.

In some embodiments, the evaluating module 34 stores a plurality of SVM models each of which corresponds to a respective one of different blood flow parameters. For example, the evaluating module 34 may store only one SVM model that corresponds to the blood flow parameter, such as a blood flow rate of 600 milliliter/minute (ml/min). Alternatively, the evaluating module 34 may store seven SVM models that respectively correspond to seven blood flow parameters, such as blood flow rates of 500, 600, 650, 750, 900, 1200 and 3500 ml/min. In some embodiments, the evaluating module 34 may store the aforementioned seven SVM models, and only one or some of the SVM models are available for use by a user based on the user's choice upon purchase of a product of the system or the method according to this disclosure.

In some embodiments, to evaluate a status of a fistula of a subject, steps 51 to 54 are performed to obtain the magnitude ratios

$$\frac{{P}_{2}}{{P}_{1}} to \frac{{P}_{n}}{{P}_{n - 1}} ,$$

and the magnitude ratios

$$\frac{{P}_{2}}{{P}_{1}} to \frac{{P}_{n}}{{P}_{n - 1}}$$

are inputted to a selected SVM model from among those SVM models that are available to the user. For example, in a scenario that the SVM model 341 that corresponds to the blood flow rate of 600 ml/min is selected, if the evaluation result outputted by the SVM model 341 is 1, it means that a blood flow rate in the fistula 91 of the subject evaluated by the method according to this disclosure is smaller than 600 ml/min; if the evaluation result outputted by the SVM model 341 is 0, it means that the blood flow rate thus evaluated is greater than or equal to 600 ml/min. The evaluation result is then outputted (e.g., displayed on a screen) to indicate the status of the fistula 91 (i.e., whether the blood flow rate is greater than 600 ml/min) for viewing by the user. In some embodiments, a medical professional may select an SVM model that corresponds to a suitable blood flow parameter from among the SVM models based on the needs during medical evaluation, and may make a determination as to whether further inspection is required based on the evaluation result.

The method for evaluating a status of a fistula according to this disclosure is related to a training process, and further includes following steps 61 and 62.

In step 61, for each unique blood flow parameter, a training data set is collected, where the training data set corresponds to the blood flow parameter and includes plural pieces of training data that respectively correspond to plural training subjects. Each of the plural pieces of training data includes a labeled category and n−1 number of magnitude ratios that are calculated in advance for the respective one of the plural training subjects.

Specifically, the blood flow parameter has to be decided first, and then the training data set that corresponds to the blood flow parameter is then collected. For example, if only one blood flow parameter that is a blood flow rate of 600 ml/min is needed, a training data set that corresponds to the blood flow parameter of 600 ml/min is collected; if two blood flow parameters that are the blood flow rates of 600 and 750 ml/min are needed, a training data set that corresponds to the blood flow rate of 600 ml/min and another training data set that corresponds to the blood flow rate of 750 ml/min are collected.

Taking the training data set that corresponds to the blood flow rate of 600 ml/min as an example, the training data set includes plural pieces of training data that respectively correspond to plural training subjects (e.g., forty-five training subjects). Each of the plural pieces of training data includes a labeled category and n−1 number of magnitude ratios. The labeled category is one of 1 and 0, where 1 indicates that a blood flow rate in the fistula of the corresponding training subject is smaller than the blood flow parameter (i.e., 600 ml/min), and 0 indicates that the blood flow rate is greater than or equal to the blood flow parameter. The n−1 number of magnitude ratios are calculated in advance by using the aforementioned steps 51 to 54 for the respective one of the plural training subjects. The labeled category is obtained based on a blood flow rate that is measured by an HD03 hemodialysis monitor produced by Transonic Systems Inc. For example, a piece of training data may be presented as

$$[ 1 , \frac{{P}_{2}}{{P}_{1}} , \frac{{P}_{3}}{{P}_{2}} , \frac{{P}_{4}}{{P}_{3}} , \frac{{P}_{5}}{{P}_{4}} , \frac{{P}_{6}}{{P}_{5}} ] ,$$

which means that the blood flow rate in the fistula of the training subject to which the piece of training data corresponds is smaller than 600 ml/min, and

$$\frac{{P}_{2}}{{P}_{1}} , \frac{{P}_{3}}{{P}_{2}} , \frac{{P}_{4}}{{P}_{3}} , \frac{{P}_{5}}{{P}_{4}} , and \frac{{P}_{6}}{{P}_{5}}$$

are the magnitude ratios obtained by performing the aforementioned steps 51 to 54.

In step 62, for each unique blood flow parameter, the training process is performed on an SVM algorithm based on the training data set that corresponds to the blood flow parameter to result in an SVM model that corresponds to the blood flow parameter.

The training process includes a cross-validation technique (e.g., k-fold cross-validation) for obtaining a predication model (i.e., the at least one SVM model). In some embodiments, k is set to be ten, that is, the plural pieces of training data of the training data set are divided into ten groups, where the ten groups may have the same number of pieces of training data, or may alternatively have different numbers of pieces of training data. Nine groups among the ten groups are used as training data for training a model, and the remaining one group is used as validation data for testing the model thus trained. This process is repeated ten times, with each of the ten groups being used exactly once as the validation data. In this way, an optimum model obtained through k-fold cross-validation may serve as the SVM model.

The principles of this disclosure are explained hereinafter.

The carrier radio wave emitted by the transmitting antenna 21 would penetrate the skin 92 of the subject, and then be reflected by a surface of the fistula 91 in the subject. Because of the Doppler effect, periodic movement of the surface of the fistula 91 due to arterial pulsation would change the frequency of the carrier radio wave reflected by the surface of the fistula 91. That is to say, the frequency of the return wave signal thus formed would be different from that of the carrier radio wave and would contain information on the periodic movement and displacements of the fistula 91. Characteristics of the return wave signal are highly related to stenosis of the fistula 91, and the reason for this can be explained based on the theory of fluid mechanics. When blood passes through a narrow segment of the fistula 91, the blood flow velocity in the narrow segment of the fistula 91 will increase and cause turbulence. Therefore, a signal detected at a position adjacent to the venous side of the fistula 91 would contain an oscillating sine wave component, resulting in a non-linear distortion of the signal. Referring to FIGS. 3 to 6, FIG. 3 illustrates a waveform 41 of the digitized detection signal for a healthy subject and FIG. 5 illustrates a spectrum 43 of the frequency spectrum information obtained from the digitized detection signal of FIG. 3 after the filtering process and the time-frequency transform; while FIG. 4 illustrates a waveform 42 of the digitized detection signal for a subject with a stenotic fistula and FIG. 6 illustrates a spectrum 44 of the frequency spectrum information obtained from the digitized detection signal of FIG. 4 after the filtering process and the time-frequency transform. It is evident that the waveform 41 of FIG. 3 is different from that of FIG. 4, and the spectrum 43 of FIG. 5 is also different from that of FIG. 6. The broken lines in FIGS. 5 and 6 are approximate curves for peaks in the spectra 43 and 44. In addition, by performing an independent T test on two groups of the magnitude ratios calculated respectively for the spectra 43 and 44 illustrated in FIGS. 5 and 6, in a scenario that the blood flow parameter is 600 ml/min, the P-value of the independent T test on the magnitude ratio of

$$\frac{{P}_{5}}{{P}_{4}}$$

is 0.008; in a scenario that the blood flow parameter is 650 ml/min, the P-value of the independent T test on the magnitude ratio of

$$\frac{{P}_{3}}{{P}_{2}}$$

is 0.035; and in a scenario that the blood flow parameter is 750 ml/min, the P-value of the independent T test on the magnitude ratio of

$$\frac{{P}_{3}}{{P}_{2}}$$

is 0.041. All of these P-values are smaller than 0.05, which means that the difference between the two groups of the magnitude ratios (i.e., a normal fistula versus a stenotic fistula) is statistically significant.

Tables 1 to 3 below present the evaluation results that are obtained by performing the method for evaluating a status of a fistula according to an embodiment of this disclosure on forty-five subjects. The evaluation results in Tables 1 to 3 are obtained with respect to the blood flow parameters of 600, 650 and 750 ml/min, respectively. The ground truth is related to blood flow rates of the subjects measured by an HD03 hemodialysis monitor produced by Transonic Systems Inc., where the ground truth is 0 when the blood flow rate thus measured is greater than the corresponding blood flow parameter, and the ground truth is 1 when the blood flow rate thus measured is smaller than or equal to the corresponding blood flow parameter.

TABLE 1
Blood flow parameter 600 ml/min

| | Evaluation result | |
|---|---|---|
| Ground truth | 0 (high) | 1 (low) |

| | | | | | |
|---|---|---|---|---|---|
| 0 | (high) | 34 | 0 | 100% | (specificity) |
| 1 | (low) | 1 | 10 | 90.9% | (sensitivity) |

TABLE 2
Blood flow parameter 650 ml/min

| | Evaluation result | |
|---|---|---|
| Ground truth | 0 (high) | 1 (low) |

| | | | | | |
|---|---|---|---|---|---|
| 0 | (high) | 29 | 0 | 100% | (specificity) |
| 1 | (low) | 1 | 15 | 93.8% | (sensitivity) |

TABLE 3
Blood flow parameter 750 ml/min

| | Evaluation result | |
|---|---|---|
| Ground truth | 0 (high) | 1 (low) |

| | | | | | |
|---|---|---|---|---|---|
| 0 | (high) | 24 | 0 | 100% | (specificity) |
| 1 | (low) | 1 | 20 | 95.2% | (sensitivity) |

According to Tables 1 to 3, the specificities are all 100% for the blood flow parameters of 600, 650 and 750 ml/min, and the sensitivities are 90.9%, 93.8% and 95.2% respectively for the blood flow parameters of 600, 650 and 750 ml/min. Therefore, it is evident that the method for evaluating a status of a fistula of a subject according to this disclosure is highly accurate.

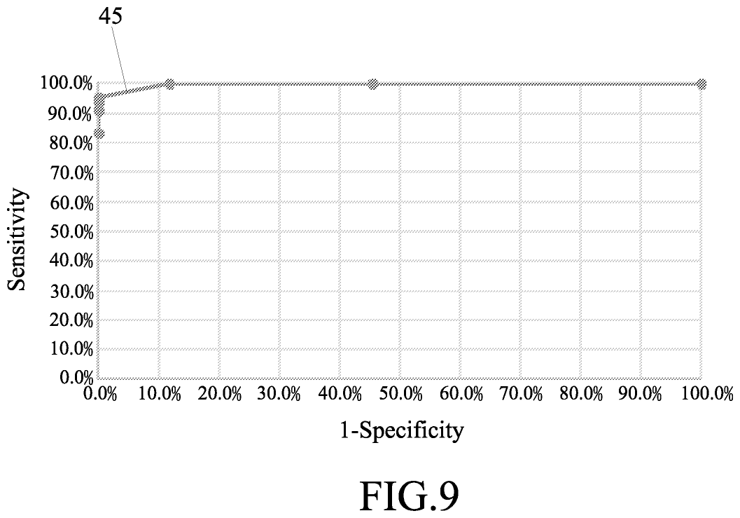

Referring to FIG. 9 and Table 4 below, a curve of receiver operating characteristic (ROC) 45 is illustrated based on the specificities and the sensitivities calculated with respect to the blood flow parameters of 500, 600, 650, 750, 900, 1200, 1800 and 3500 ml/min. An area under the curve (AUC) of ROC 45 is 0.997, which is close to one, and it can be proved that the method according to the disclosure achieves high prediction accuracy.

TABLE 4

Blood flow parameter

| | 500 | 600 | 650 | 750 |
|---|---|---|---|---|
| Specificity | 100% | 100% | 100% | 100% |
| Sensitivity | 83.3% | 90.9% | 93.8% | 95.2% |

Blood flow parameter

| | 900 | 1200 | 1800 | 3500 |
|---|---|---|---|---|
| Specificity | 88.2% | 54.5% | 0% | 0% |
| Sensitivity | 100% | 100% | 100% | 100% |

In summary, the system and method for evaluating a status of a fistula according to this disclosure at least have the following effects.

By emitting the carrier radio wave and receiving the return wave signal, and by performing the time-frequency transform on the digitized detection signal, the frequency spectrum information is obtained. At least one magnitude of harmonic and at least one magnitude ratio are then calculated based on the frequency spectrum information. By using a machine learning model based on the at least one magnitude ratio, an evaluation result which indicates the status of the fistula may be generated.

The cost incurred in such an approach is much lower than purchasing the HD03 hemodialysis monitor produced by Transonic Systems Inc., and no consumable is required. In addition, the approach adopted by the disclosure is non-intrusive, and is convenient to use. Therefore, the method and the system for evaluating a status of a fistula according to the disclosure are suitable for regular check-ups in hospitals and home heath care.

When the evaluation result generated by the method or the system according to the disclosure indicates an abnormal status of the fistula (e.g., the blood flow rate is lower than a threshold, such as 600 ml/min), the HD03 hemodialysis monitor produced by Transonic Systems Inc. or other instruments may then be adopted to perform more accurate assessment. In this way, stenosis in the fistula may be detected in the early phase before occurrence of vascular obstruction, and medical professionals may perform appropriate treatment (e.g., arteriovenous fistula angioplasty) in time. Hence, effect of hemodialysis may be improved, re-admission rate may be reduced, medical expenditure may be reduced, and quality of life may be enhanced.

Moreover, according to the aforementioned validation results presented by Tables 1 to 4 and FIG. 9, the specificities are all 100% for blood flow parameters of 600, 650 and 750 ml/min; the sensitivities are 90.9%, 93.8% and 95.2% respectively for the blood flow parameters of 600, 650 and 750 ml/min;

the sensitivities are all 100% for blood flow parameters of 900, 1200, 1800 and 3500 ml/min; and the AUC is 0.997. Therefore, it is evident that the system and the method according to this disclosure are able to achieve high prediction accuracy.

In the description above, for the purposes of explanation, numerous specific details have been set forth in order to provide a thorough understanding of the embodiment(s). It will be apparent, however, to one skilled in the art, that one or more other embodiments maybe practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects, and that one or more features or specific details from one embodiment may be practiced together with one or more features or specific details from another embodiment, where appropriate, in the practice of the disclosure.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A system for evaluating a status of a fistula of a subject, comprising:
a radio device including
a transmitting antenna,
a receiving antenna,
a transmitting module that is configured to cooperate with said transmitting antenna to emit a carrier radio wave toward the fistula, and
a receiving module that is configured to receive, via said receiving antenna, a return wave signal that is formed through reflection of the carrier radio wave by the fistula, and to output a transmission signal that is generated based on the return wave signal; and
an evaluating device including
a communication module that is in signal connection with said receiving module, and that is configured to receive the transmission signal, and to recover a digitized detection signal from the transmission signal,
a time-frequency transform module that is connected to said communication module, and that is configured to receive the digitized detection signal, to perform a time-frequency transform on the digitized detection signal to result in frequency spectrum information, and to output the frequency spectrum information,
a calculating module that is connected to said time-frequency transform module, and that is configured to receive the frequency spectrum information, to determine, from the frequency spectrum information, a frequency that corresponds to a greatest magnitude as a fundamental frequency, to calculate at least one magnitude of harmonic, and to calculate at least one magnitude ratio that is one of following items:
a ratio of one of the at least one magnitude of harmonic to the greatest magnitude that corresponds to the fundamental frequency;
a ratio of one of the at least one magnitude of harmonic to another one of the at least one magnitude of harmonic; and
a combination thereof,
where the at least one magnitude of harmonic is a peak magnitude for at least one harmonic frequency band with respect to the fundamental frequency, and
an evaluating module that is in signal connection with said calculating module, and that is configured to receive the at least one magnitude ratio thus calculated, to generate an evaluation result by using a machine learning model based on the at least one magnitude ratio, and to output the evaluation result that indicates the status of the fistula.

2. The system as claimed in claim 1, wherein:
said calculating module is configured to calculate the at least one magnitude of harmonic that includes n−1 number of magnitudes of harmonics, where the n−1 number of magnitudes of harmonics are peak magnitudes respectively for n−1 number of harmonic frequency bands with respect to the fundamental frequency, and to calculate the at least one magnitude ratio that includes n−1 number of magnitude ratios based on an expression of
$$\frac{{P}_{i}}{{P}_{i - 1}} ,$$
where i represents an integer variable that starts from two to n, n represents a positive integer greater than one, Pᵢ represents an i^{th} magnitude of harmonic of the n−1 number of magnitudes of harmonics, Pᵢ₋₁ represents an (i−1)^{th} magnitude of harmonic of the n−1 number of magnitudes of harmonics, and P₁ represents the greatest magnitude that corresponds to the fundamental frequency; and
said evaluating module is configured to generate the evaluation result by using the machine learning model based on the n−1 number of magnitude ratios, and to output the evaluation result.

3. The system as claimed in claim 2, wherein:
said time-frequency transform module is configured to perform a filtering process on the digitized detection signal to result in a filtered signal that is in a specific passband, and to perform the time-frequency transform on the filtered signal to result in the frequency spectrum information; and
said calculating module is configured to select the n−1 number of harmonic frequency bands from within the specific passband before calculating the peak magnitudes respectively for the n−1 number of harmonic frequency bands.

4. The system as claimed in claim 2, wherein
said evaluating module is configured to store a support vector machine (SVM) model to serve as the machine learning model, and to generate the evaluation result by using the SVM model based on the n−1 number of magnitude ratios.

5. A method for evaluating a status of a fistula of a subject, comprising:
emitting a carrier radio wave toward the fistula;
receiving a return wave signal that is formed through reflection of the carrier radio wave by the fistula, and outputting a transmission signal that is generated based on the return wave signal;
recovering a digitized detection signal from the transmission signal;
performing a time-frequency transform on the digitized detection signal to result in frequency spectrum information, and outputting the frequency spectrum information;
determining, from the frequency spectrum information, a frequency that corresponds to a greatest magnitude as a fundamental frequency, calculating at least one magnitude of harmonic, and calculating at least one magnitude ratio that is one of following items:
a ratio of one of the at least one magnitude of harmonic to the greatest magnitude that corresponds to the fundamental frequency;
a ratio of one of the at least one magnitude of harmonic to another one of the at least one magnitude of harmonic; and
a combination thereof,
where the at least one magnitude of harmonic is a peak magnitude for at least one harmonic frequency band with respect to the fundamental frequency; and
generating an evaluation result by using a machine learning model based on the at least one magnitude ratio, and outputting the evaluation result that indicates the status of the fistula.

6. The method as claimed in claim 5, wherein:
calculating at least one magnitude of harmonic includes calculating n−1 number of magnitudes of harmonics, where the n−1 number of magnitudes of harmonics are peak magnitudes respectively for n−1 number of harmonic frequency bands with respect to the fundamental frequency;
calculating at least one magnitude ratio includes calculating n−1 number of magnitude ratios based on an expression of
$$\frac{{P}_{i}}{{P}_{i - 1}} ,$$
where i represents an integer variable that starts from two to n, n represents a positive integer greater than one, Pᵢ represents an i^{th} magnitude of harmonic of the n−1 number of magnitudes of harmonics, Pᵢ₋₁ represents an (i−1)^{th} magnitude of harmonic of the n−1 number of magnitudes of harmonics, and P₁ represents the greatest magnitude that corresponds to the fundamental frequency; and
generating an evaluation result includes generating the evaluation result by using the machine learning model based on the n−1 number of magnitude ratios.

7. The method as claimed in claim 6, wherein calculating at least one magnitude of harmonic includes:
multiplying the fundamental frequency by two to n to obtain n−1 number of harmonics;
for each of the n−1 number of harmonics, determining a frequency band that is a specific range of frequencies with a center frequency being the harmonic is determined as a respective one of the n−1 number of harmonic frequency bands; and
for each of the n−1 number of harmonic frequency bands, determining a greatest magnitude among magnitudes that correspond to frequencies in the specific range as the peak magnitude for the harmonic frequency band.

8. The method as claimed in claim 6, further comprising:
for a unique blood flow parameter, collecting a training data set that corresponds to the blood flow parameters and that includes plural pieces of training data which respectively correspond to plural training subjects, where each of the plural pieces of training data includes a labeled category and n−1 number of magnitude ratios that are calculated in advance for the respective one of the plural training subjects; and
for the unique blood flow parameter, performing a training process on a support vector machine (SVM) algorithm based on the training data set that corresponds to the blood flow parameter to result in an SVM model that corresponds to the blood flow parameter.
